(19) United States
(12) Patent Application Publication
(10) Pub. No.: US 2026/0365817 A1
(43) Pub. Date: Nov. 19, 2026

(54) POLYOL (OLEOPOL) PRODUCTION FROM RENEWABLE RESOURCES

(71) Applicant: RELAB ARGE TEKNOLOJILERI ANONIM SIRKETI, Istanbul (TR)

(72) Inventors: Recep EROGLU, Istanbul (TR); Emre EROGLU, Istanbul (TR); Esref Hilmi ACIK, Istanbul (TR); Gokhan CAYLI, Istanbul (TR)

(73) Assignee: RELAB ARGE TEKNOLOJILERI ANONIM SIRKETI, Istanbul (TR)

(21) Appl. No.: 18/995,986

(22) PCT Filed: Jul. 8, 2023
(86) PCT No.: PCT/TR2023/050666

§ 371 Date: Jan. 17, 2025

(30) Foreign Application Priority Data
Jul. 22, 2022 (TR) 2022/011717

(51) Int. Cl. C08J 11/26; C07C 67/26; C08J 11/24

(52) U.S. Cl. CPC C08J 11/26; C07C 67/26; C08J 11/24; C08J 2391/00

(57) ABSTRACT

A method for producing polyols (oleopol, oleomilk) from renewable resources is provided. The method includes making waste vegetable or animal oils ready for use by epoxying; extracting oleic acid from waste pomace oil by a two-step reaction; adding acetic acid or lactic acid and/or the oleic acid to an epoxide soybean oil, refluxing it in a reflux cooler, then evaporating the excess acetic acid or lactic acid and obtaining polyols (oleopol, oleomilk).

### CROSS REFERENCE TO THE RELATED APPLICATIONS

This application is the national phase entry of International Application No. PCT/TR2023/050666, filed on Jul. 8, 2023, which is based upon and claims priority to Turkish Patent Application No. 2022/011717, filed on Jul. 22, 2022, the entire contents of which are incorporated herein by reference.

### TECHNICAL FIELD

The invention relates to a method for producing polyols (oleopol, oleomilk) from renewable resources.

### BACKGROUND

Polyurethanes, one of the polymers with various uses, have two raw materials consisting of isocyanate and polyol. Polyurethanes are obtained as a result of the reaction of these raw materials. In these reactions, isocyanates such as HDI (hexamethylene diisocyanate), MDI (methylene diphenylenediisocyanate), TDI (toluene diisocyanate) and IDI (isophorone diisocyanate) can be used as isocyanate components. There are limited producers in the world due to its extreme toxicity and the difficulty of the production procedure. The polyol component can be small simple molecules such as monoethyleneglycol, propylene glycol or structures attached to a long polymeric chain. In these structures, they are usually referred to by names such as polyester, polyamide polyol, polyester polyol. Polyols can be grouped into various types according to their usage areas. It reacts with isocyanates to make foam insulation for mattresses, refrigerators and freezers, polyurethanes for household and automotive seats, polyurethanes for synthetic leather for clothing, elastomeric shoe soles, fibers and adhesives.

Polyols contain multiple hydroxyl groups and are reactive raw materials that react with isocyanate (NCO) groups to form polyurethane structures. Polyols are substances with a wide range of molecular weights and can therefore exist in different physical forms (solid, liquid).

### SUMMARY

The present invention relates to the production of polyol (oleopol, oleomilk), which meets the above-mentioned requirements, eliminates all disadvantages and brings some additional advantages.

The main purpose of the invention is to obtain polyols by an environmentally friendly process using raw materials from renewable resources. The raw materials used in the invention are substances with ethylenic double bonds. The most well-known of these are materials called triglycerides. Triglycerides are also known as fats. The oils subject to the invention are waste vegetable or animal fats. Substances with these ethylenic double bonds, especially oils, are epoxylated and made ready for use. These oils are reacted with the right combinations to obtain polyols with the desired properties.

In the synthesis of polyols produced within the scope of the invention, acids with groups such as carboxyl, sulfoxyl, which can be obtained naturally, and epoxide compounds obtained naturally in nature (vernonia oil) or materials containing double bonds and converted into epoxy derivatives by an appropriate procedure are used.

In the invention, polyol (oleopol, oleomilk), which is one of the raw materials to be used in the production of polyurethane as a result of the trials carried out, is obtained from renewable sources (from industrial olive oil waste, pirin and various vegetable and animal oils), contains approximately 3.5-6-OH (hydroxyl) groups per triglyceride, gives the desired physical and chemical properties and is made ready for use.

The structural and characteristic features and all advantages of the invention will be more clearly understood with the detailed description given below and therefore the evaluation should be made by taking this detailed description into consideration.

### DETAILED DESCRIPTION OF THE EMBODIMENTS

In this detailed description, the preferred embodiments of the inventive method are described only for a better understanding of the subject matter.

The invention relates to a method for producing polyols (oleopol, oleomilk) from renewable resources. In this production method, preferably waste vegetable oils are epoxied and made ready for use. In the next process step, oleic acid is obtained from waste pomace oil. For this purpose, pomace oil is saponified using a base in the first step and then the soap is acidified and converted into free fatty acid (in this case oleic acid). In the first process, acetic acid and/or oleic acid is added to the epoxidized soybean oil obtained in the first process and refluxed in a reflux cooler, the acetic acid is evaporated and polyol (oleopol) can be produced. Depending on the amounts of oleic acid and acetic acid used, polyol (oleopol or oleomilk) with different percentages of acid composition can be obtained. In this way, polyols (oleopol, oleomylk) are obtained step by step from natural raw materials with ethylenic double bonds, especially vegetable oils.

Examples of the method of obtaining polyol (oleopol, oleomilk) realized within the scope of the invention are given below. These examples are illustrative of the application and effectiveness of the invention without any restrictive meaning.

### Example 1. Epoxy Oil Production

Add 50 grams of waste oil to a 250 milliliter balloon with a flat bottom. Add 100 milliliters of methylene chloride and cool the mixture in an ice bath. Then 24 milliliters of formic acid is added to this solution and the mixture is continued to be stirred for another half hour. At the end of this time, 18 milliliters of hydrogen peroxide is added to the mixture drop by drop over half an hour. Leave the mixture to stir in an ice bath for 1 night. At the end of the day, the mixture is washed with distilled water. If there is any water left in the mixture, add sodium sulfate to remove the water.

### Example 2. Oleic Acid Production

Put 100 grams of waste pomace oil from industrial olive oil production into a 250 milliliter balloon with a flat bottom. Add 30 g of this oil to 50% sodium hydroxide solution and stir for 2 hours to make soap. The resulting soap is oleic acid soap. Then take this soap and add 60 milliliters of concentrated (36-37%) chloric acid and 250 milliliters of distilled water. The mixture is heated at 60-70° C. for 1 night. When there is phase separation, the upper part is removed and oleic acid is obtained.

### Example 3. Synthesis Of Eso-Acetic Acid Condensate Oleopol

Put 100 grams of epoxidized soybean oil-ESO- into a 250 milliliter balloon with a flat bottom. Add 90 milliliters of acetic acid and boil at 100-140° C. for about 8 hours in a refluxer. At the end of the time, excess acetic acid is evaporated in a rotary mixer.

### Example 4. Synthesis Of 30% Oleopol

Put 100 grams of epoxidized soybean oil—ESO—into a 250 milliliter balloon with a flat bottom. Add 7.6 grams of oleic acid and 24 grams of acetic acid and reflux at 100-140° C. for about 8 hours with a reflux cooler. At the end of the time, the acetic acid is evaporated in a rotary mixer.

### Example 5. Synthesis Of 50% Oleopol

Put 100 grams of epoxidized soybean oil-ESO- into a 250 milliliter balloon with a flat bottom. Add 12.5 grams of oleic acid and 12.5 grams of acetic acid and reflux at 100-140° C. for about 8 hours with a reflux cooler. At the end of the time, the acetic acid is evaporated in a rotary mixer.

### Example 6. Synthesis Of Eso-Lactic Acid Condensate Oleomycin

Put 100 grams of epoxidized soybean oil-ESO- into a 250 milliliter balloon with a flat bottom. Add 30 grams of lactic acid and reflux at 100-140° C. for about 8 hours with a reflux cooler. At the end of the time, the lactic acid is evaporated in a rotary mixer.

### Example 7. Synthesis Of 50% Oleomycin

Put 100 grams of epoxidized soybean oil—ESO—into a 250 milliliter balloon with a flat bottom. Add 12.5 grams of oleic acid and 12.5 grams of lactic acid and reflux at 100-140° C. for about 8 hours with a reflux cooler. At the end of the time, the lactic acid is evaporated in a rotary mixer.

What is claimed is:

1. A method for producing polyol from renewable resources, comprising
making waste vegetable or animal oils ready for use by epoxying,
extracting oleic acid from a waste pomace oil by a two-step reaction,
adding acetic acid or lactic acid and/or the oleic acid to an epoxide soybean oil to obtain a mixture, refluxing the mixture in a reflux cooler, then evaporating excess acetic acid or lactic acid and obtaining the polyol.

2. The method according to claim 1, comprising a step of obtaining polyol in different percentages, comprising 30% and 50%, according to amounts of the acetic acid or the lactic acid and/or the oleic acid added.
